(19) United States
(12) Patent Application Publication
(10) Pub. No.: US 2026/0102457 A1
(43) Pub. Date: Apr. 16, 2026

(54) ANTICOAGULANT-PEPTIDE NANOGRANULES FOR THROMBOSIS-ACTUATED ANTICOAGULATION

(71) Applicant: The Penn State Research Foundation, University Park, PA (US)

(72) Inventor: Scott Hammond MEDINA, State College, PA (US)

(21) Appl. No.: 19/114,890

(22) PCT Filed: Sep. 26, 2023
(86) PCT No.: PCT/US2023/075081

§ 371 Date: Mar. 25, 2025

Related U.S. Application Data
(60) Provisional application No. 63/410,078, filed on Sep. 26, 2022.

(51) Int. Cl. A61K 38/10; A61K 31/727; A61P 7/02

(52) U.S. Cl. CPC A61K 38/10; A61K 31/727; A61P 7/02

(57) ABSTRACT

Provided are compositions and uses of the compositions for anti-coagulation purposes. The compositions comprise a peptide component and an anticoagulant. The peptide has the sequence KRWIWWRRHWVVW (SEQ ID NO:1) and optionally comprises an amido group at the C-terminus of the peptide. The anticoagulant may be any form of heparin. Subcutaneous delivery formations are provided that improve the efficacy of the heparin.

### CROSS REFERENCE TO RELATED APPLICATIONS

This application claims the benefit of U.S. provisional patent application No. 63/410,078, filed Sep. 26, 2022, the entire disclosure of which is incorporated herein by reference.

### SEQUENCE LISTING

The instant application contains a Sequence Listing which has been submitted in xml format and is hereby incorporated by reference in its entirety. Said .xml copy was created on Sep. 22, 2023, is named “PSU_MAD1_Medina_sequence.xml” and is 1,832 bytes in size.

### RELATED INFORMATION

Since the discovery of its anticoagulant properties a century ago, heparin has been widely used to prevent and treat blood clots. Clinically, heparin is administered by intravenous infusion or subcutaneous injection, with dosing carefully adjusted for the patient's body weight and clotting sensitivity. Patient variability, user error, and infuser malfunction can all lead to heparin overdosing. Additionally, its short plasma half-life (t_{1/2}=˜1 hour) requires maintenance administration of heparin every 1 to 4 hours, significantly increasing the risk of dosing errors. Major adverse effects include severe thrombocytopenia, hemorrhage, and death, particularly in small children and pregnant women.^{[1-4]} Developing subcutaneously injectable materials that controllably release heparin may allow for less frequent dosing of patients and reduce the incidence of overdose complications. In addition, new shelf-stable and easy-to-administer heparin formulations may also open opportunities for their use in non-clinical settings (e.g., battlefield).

However, the polyanionic and polydisperse nature of heparin has made developing alternative delivery systems a formidable challenge. Several materials-enabled strategies are currently being developed for oral and pulmonary heparin delivery, the majority of which rely on hydrogel, nanoparticle, or nanofiber vehicles.^{[5-8]} Indeed, the use of these delivery technologies reduces side effects and enables temporal control over heparin release. Nevertheless, rapid metabolism and clearance in the gastrointestinal and respiratory tracts has led to only a small fraction of active drug (<3-5%) reaching the blood stream.^{[8]} Therefore, subcutaneous depots of heparin that can gradually diffuse into circulation to sustain therapeutically relevant plasma concentrations and enable ‘on-demand’ anticoagulant functionality are needed. The present disclosure is pertinent to this need.

### BRIEF SUMMARY

The present disclosure provides anti-coagulant nanogranules that can be compacted to form a subcutaneously injectable material, such as a paste. The granular material is produced by templated organization of an anti-coagulant such as heparin with cationic protofibrillar nanostructures formed via self-assembly of the tryptophan-rich peptide MAD1. Clotting assays show that heparin-MAD1 granules (HMGs) initially sequester heparin to inhibit its anticoagulant activity during circulation. Under the high fluid sheer forces experienced around thrombi, however, the HMG constituents are mechanically decoupled to ‘turn-on’ heparin's antithrombotic function. Further, HMGs can be clustered and compacted under centrifugation to form an injectable material such as a gel or paste that can be readily administered subcutaneously in animals. Release studies show that physiologic erosion of the injected material liberates bioactive heparin-MAD1 nanogranules, which subsequently bind activated platelets in the growing thrombus. In vivo studies show that these unique properties converge to allow HMG depots to sustain heparin serum concentrations for an order of magnitude longer, and provide prolonged and controlled anticoagulant activity, relative to control animals receiving a bolus dose of free heparin given intravenously or subcutaneously.

### BRIEF DESCRIPTION OF FIGURES

FIG. 1. Formation of granular nanoclusters via heparin-MAD1 assembly. (a) Scanning electron microscopy (SEM) images of heparin-MAD1 nanogranule formation and clustering. From left to right, formation of discrete heparin-MAD1 nanoparticles is followed by their aggregation to form micron scale clusters, which go on to accrete and yield dense plate structures. Procedural assembly diagram shown above each SEM image. Scale bars=1 μm. (b) Dynamic light scattering analysis of heparin-MAD1 assemblies. Highlighted features correspond to individual nanoparticles and aggregated clusters. The final stage, granulated plates, are too large to be detected by DLS. (c) Zeta potential measurements of HMGs. Imaging and optical analyses performed at n≥3, with representative results shown.

FIG. 2. Mechanistic investigation of heparin-MAD1 assembly to form HMGs. (a) Circular dichroism (CD) spectra of HMGs at MAD1-to-heparin weight ratios of 1:0.1 (left), 1:1 (middle) and 1:10 (right). Ellipticity profile of free MAD1 shown in open circles for each comparison. Mechanistic schematics of heparin (orange) intercalation into self-assembled MAD1 protofibrils (grey) are shown below each spectra. (b) Emission spectra of ThT in the absence (blank, black) or presence of MAD1 (red), heparin (blue), or HMGs (green). (c) HPLC-based agglutination assays following incubation of the MAD1 peptide in the absence (black) or presence (+ compound) of glucose, mannose, bovine serum albumin (BSA), hyaluronic acid (HA) and heparin. Experiments performed at n≥3, with representative results shown.

FIG. 3. HMG anticoagulant and hemolytic activity in recalcified blood samples. (a) Clotting time of blood treated with heparin (blue), MAD1 (red) or the HMG complex (green) at varying concentrations of equivalent heparin. MAD1 samples were treated with the free peptide at concentrations matching that encumbered within HMGs, and reported as the corresponding equivalent heparin concentration to allow for uniform comparison. (b) Clotting time of blood treated in the absence (blank) or presence of heparin, MAD1 or HMGs at an equivalent heparin concentration of 2 μg/mL. (c) Clotting time of blood treated with HMGs before (open green bar) and after (hatched green bar) decoupling of heparin under ultrasound (US; 40 kHz, 240 W). Free heparin (blue) included as a control. All samples treated with 2 μg/mL of equivalent heparin. Experiments performed at n=3 and reported as mean±std. dev., with individual points shown where appropriate. Statistical significance determined using Student's t-test and represented as n.s.=not significant, *** p<0.001.

FIG. 4. Cellular localization, uptake and bioactivity of HMGs. (a) Representative confocal micrographs of MAD1 (green), heparin (cyan) and HMG binding to activated platelets (red). Individual fluorescence channels and merged images are shown for each treatment condition. Scale bar=100 μm. (b) Magnified image from region of interest in panel a (white dashed square) demonstrating agglutination of activated platelets by MAD1 fibrillar assemblies (see white arrows). Scale bar=20 μm. (c) Flow cytometry histograms of activated platelets before (black) and after (green) incubation with FITC-labeled MAD1. (d) Percent erythrocyte hemolysis at varying HMG concentrations, reported as equivalent heparin concentration. Triton X-100 (TX, black) included as a positive control. (e) Confocal micrographs of murine RAW264.7 macrophages treated with HMGs (green) at varying incubation times. Scale bar=100 μm. (f) Stacked flow cytometry histograms RAW264.7 macrophages before (black) and after (green) treatment with fluorescent HMGs at the indicated incubation time point. Experiments performed at n=3 and reported as mean±std. dev., with representative results shown where appropriate.

FIG. 5. Preparation of compacted HMG injectable gels. (a) Schematic of the centrifugal processing of HMG solution to yield a condensed paste. Granules densely organize via spherical packing. (b) Scanning electron micrograph of the HMG paste. Fissures between the material are artifacts caused by the dehydration processing for SEM analysis. See rehydrated sample in Scale bars=20 μm. (c) Time-dependent release of fluorescently-labeled heparin (blue) and MAD1 (red) from the HMG paste under physiologic conditions. (d) Fluorescent micrograph of the HMG paste prepared using FITC-MAD1. Scale bar=200 μm. Inset. Magnified region of interest (dashed box) showing the release of micron-scale granules during paste erosion (white arrows). Scale bar=50 μm. Imaging and optical analyses performed at n≥3 and reported as mean±sem., or representative images shown.

FIG. 6. In vivo performance of HMGs. (a) Time-dependent heparin plasma concentrations following its delivery as either an intravenous (heparin_{i.v.}) or subcutaneous (heparin_{s.c.}) bolus, or delivered from subdermal HMG depots (HMG_{s.c.}). (b) Blood clotting time at 0.5-24 hours following heparin delivery in the heparin_{i.v.}, heparin_{s.c.}, HMG_{s.c.} groups. Clotting times were determined from thrombus viscoelasticity measurements of recalcified blood samples. Dashed line represents nominal clotting time for blood collected from untreated animals (3.5 min.). (c) Heatmaps illustrating time-dependent heparin accumulation within liver, kidney and spleen tissues. Fluorescence data was normalized to untreated control tissues and reported in arbitrary fluorescence units.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all steps and compositions of matter described herein in the text and figures of this disclosure, including all such steps individually and in all combinations thereof, and includes all compositions of matter and methods of using the same as described herein.

Throughout this application, unless stated differently, the singular form encompasses the plural and vice versa. All sections of this application, including any supplementary sections or figures, are fully a part of this application.

The disclosure includes all amino acid sequences described herein, and variants thereof.

This application relates in part to U.S. Pat. Nos. 11,458,099, 11,571,454, and 11,717,482, the entire disclosures of each of which are incorporated herein by reference.

In one embodiment the disclosure provides a composition for use in inhibiting blood clotting, the composition comprising an anionic and cationic component, wherein at least one of the components comprises an anticoagulant agent. In an embodiment the anticoagulant agent is the anionic component, which may be heparin. In an embodiment the cationic component comprises a peptide. In an embodiment the peptide comprises or consists of the amino acid sequence KRWHWWRRHWVVW (SEQ ID NO:1), and wherein the peptide optionally comprises an amido group at the C-terminus of the peptide.

In an embodiment the peptide and the anticoagulant may be provided in the form of subcutaneously deliverable anticoagulant-peptide nanogranules that allow for long-lasting anticoagulant bioavailability in the circulatory system, while enabling on-demand activation of the anticoagulant's anticoagulant effects in the thrombus microenvironment. Biophysical studies described herein demonstrate this responsive behavior is due to sequestration of anticoagulant within self-assembling peptide nanofibrils and its mechanically actuated decoupling to elicit antithrombotic effects at the clotting site. In vivo studies show these unique properties converge to allow subcutaneous nanogranule depots to extend anticoagulant serum concentrations for an order of magnitude longer than standard dosing regimens, while enabling prolonged and controlled anticoagulant activity. This biohybrid delivery system demonstrates a scalable platform for the development of safer, easier to administer and more effective antithrombotic nanotechnologies.

In an embodiment, the disclosure provides a complex comprising a peptide and an agent that exhibits anticoagulation properties. In one embodiment, the peptide comprises an amido group (—NH2) at the C-terminus. In embodiments, a peptide of this disclosure comprises or consists of the amino acid sequence KRWHWWRRHWVVW (SEQ ID NO: 1). In an embodiment, the peptide comprises an amido group at the C-terminus and thus comprises KRWHWWRRHWVVW-NH2 (SEQ ID NO:1) (MAD1).

In embodiments the described peptide is combined with an anticoagulant that is heparin. Heparin is known in the art and is commercially available. In embodiments, the heparin comprises unfractionated heparin. In embodiments, heparin used in embodiments of this disclosure has the structure:

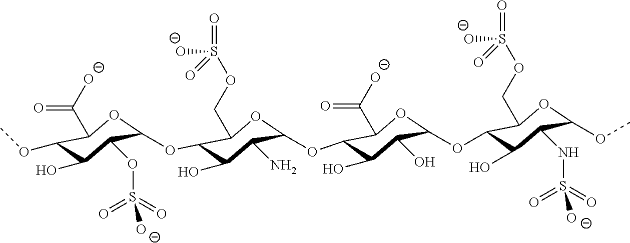

The disclosure includes use of heparin and its low-molecular-weight derivatives, such as enoxaparin, dalteparin, and tinzaparin.

In embodiments, an effective amount of a described composition is administered to an individual in need thereof. In embodiments, an effective amount is an amount that reduces one or more signs or symptoms of a disease or disorder, and/or reduces the severity of the disease or disorder. An effective amount may also inhibit or prevent the onset of a condition. A precise dosage can be selected by the individual physician in view of the patient to be treated. Dosage and administration can be adjusted to provide sufficient levels of the described composition to maintain the desired effect. Additional factors that may be taken into account include age, weight, and gender of the patient, desired duration of treatment, method of administration, time and frequency of administration, drug combination(s), reaction sensitivities, and the like.

The described compositions can be administered to an individual in need thereof using any suitable route, examples of which include intravenous, intramuscular, intraperitoneal, subcutaneous, oral, topical, or inhalation routes. In embodiments, a described composition is implanted subcutaneously, and may be within any suitable matrix. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated. In embodiments a described composition is introduced to an individual as a subcutaneously administered matrix in which, for example, heparin is present. In embodiments, the heparin remains a component of a particle until the particle encounters fluid sheer forces in the circulatory system of an individual.

In embodiments, the individual in need of a composition of this disclosure has been diagnosed with or is suspected of having a disease or a condition wherein blood clotting is involved. In embodiments, the individual is in need or would benefit from receipt of an anticoagulant. In embodiments, the individual is undergoing or is scheduled to undergo treatment for a blood or organ condition. In embodiments, the individual is undergoing or is a candidate for a surgical procedure. In embodiments, the individual has been diagnosed with or is suspected of having any of disseminated intravascular coagulation, venous thrombosis, deep-vein thrombosis, pulmonary embolism, peripheral arterial embolism, a stroke, acute coronary syndrome, atrial fibrillation, a heart attack, or unstable angina. In embodiments the individual is undergoing prolonged bed rest or hemofiltration, or has an indwelling catheter.

In embodiments, administering a described composition exhibits an improved activity relative to administration of a control composition.

In embodiments, an effective amount of a described composition is administered to an individual in need thereof. In embodiments, an effective amount is an amount that reduces one or more signs or symptoms of a disease or disorder, and/or reduces the severity of the disease or disorder. An effective amount may also inhibit or prevent the onset of a condition. A precise dosage can be selected by the individual physician in view of the patient to be treated. Dosage and administration can be adjusted to provide sufficient levels of the described composition to maintain the desired effect. Additional factors that may be taken into account include age, weight, and gender of the patient, desired duration of treatment, method of administration, time and frequency of administration, drug combination(s), reaction sensitivities, and the like.

The described compositions can be administered to an individual in need thereof using any suitable route, examples of which include subcutaneous, intravenous, intramuscular, intraperitoneal, oral, topical, or inhalation routes. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated. In embodiments a described composition is introduced to an individual as a subcutaneously administered matrix in which, for example, heparin is present. In embodiments, the heparin remains a component of a particle until the particle encounters fluid sheer forces in the circulatory system of an individual. In an embodiment the matrix is provided in a flowable form, such as a paste.

In embodiments, the individual in need of a composition of this disclosure has been diagnosed with or is suspected of having a disease or a condition wherein blood clotting is involved. In embodiments, the individual is in need or would benefit from receipt of an anticoagulant. In embodiments, the individual is undergoing or is scheduled to undergo treatment for a blood or organ condition. In embodiments, the individual is undergoing or is a candidate for a surgical procedure. In embodiments, the individual has been diagnosed with or is suspected of having any of disseminated intravascular coagulation, venous thrombosis, deep-vein thrombosis, pulmonary embolism, peripheral arterial embolism, a stroke, acute coronary syndrome, atrial fibrillation, a heart attack, or unstable angina. In embodiments the individual is undergoing prolonged bed rest or hemofiltration, or has an indwelling catheter.

The following Examples are intended to illustrate but not limit the disclosure.

### Example 1

### Assembly and Characterization of HMGs

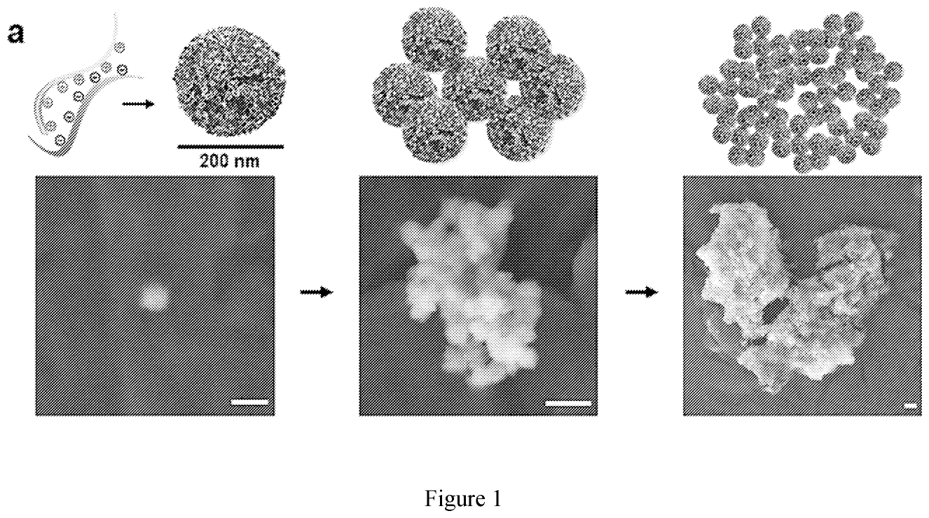

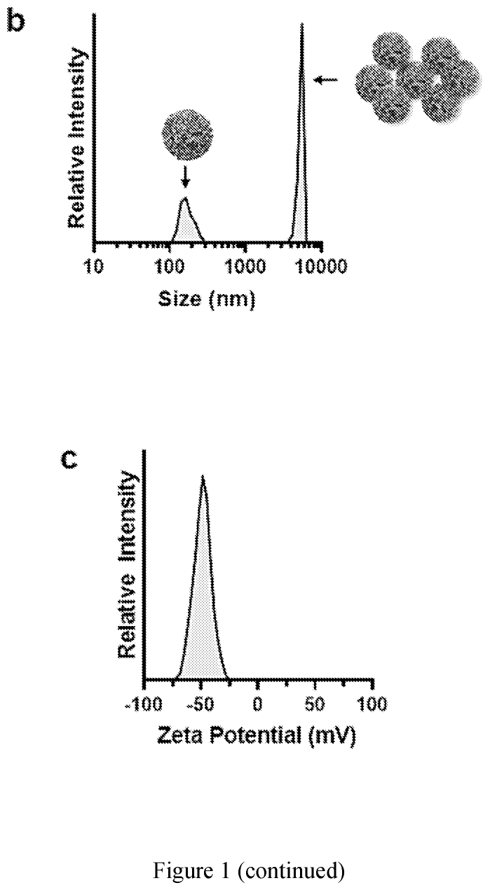

Heparin is a naturally occurring glycosaminoglycan that binds to and activates the inhibitory enzyme antithrombin, leading to inactivation of thrombin, factor Xa and other pro-clotting proteases. In addition to its anticoagulant properties, heparin can scavenge iron to inhibit the replication of Mycobacterium tuberculosis, the causative bacterial agent of Tuberculosis, within infected human macrophages.^{[10]} This activity motivated us to test the anti-TB efficacy of heparin in combination with a de novo designed antitubercular peptide recently developed by our lab, named MAD1 (mycomembrane associated disruption 1).^{[9, 11]} During these studies, we observed that co-incubation of heparin and MAD1 in aqueous media led to the formation of micron-scale aggregates, which were otherwise absent in the monotherapies. Scanning electron microscopy performed at various time intervals during heparin-MAD1 assembly indicated this was due to the initial formation of spherical polyplexes (FIG. 1a, left), approximately 200 nm in diameter. These granules subsequently organize into micron-scale clusters (FIG. 1a, middle) and later compact to form plates (FIG. 1a, right). Light scattering analyses suggest this is a dynamic assembly process, with individual HMGs existing in equilibrium with the micro- and macro-assembled states (FIG. 1b). A negative zeta potential of the granules indicates anionic heparin molecules predominantly decorate the surface (FIG. 1c). In isolation, MAD1 organizes into protofibrillar nanostructures due to intermolecular tryptophan-tryptophan pairing.^{[9]} This suggests that MAD1 peptide fibrils may nucleate the electrostatic assembly of heparin at the fibril surface to form the observed anionic HMG particulates.

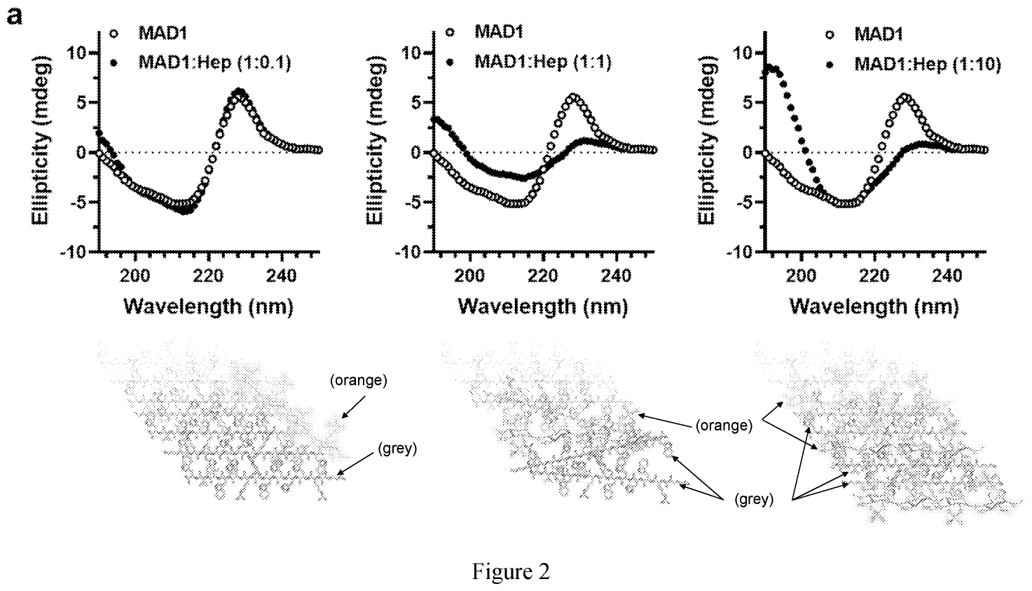

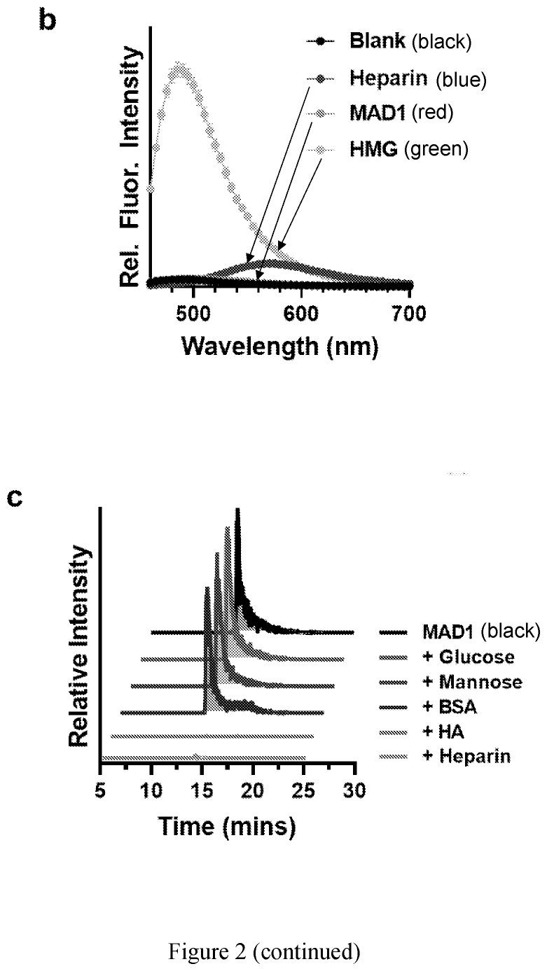

To test this assertion, we performed circular dichroism (CD) spectroscopy on MAD1 assemblies in the presence of increasing heparin concentrations (FIG. 2a). In the absence of heparin, MAD1 self-assembly evolves a CD spectrum indicative of a tryptophan zipper,^{[12, 13]} with putative exciton-coupled bands at 212 and 228 nm (see open circles in FIG. 2a). Here, the maximum at 228 nm results from interactions between tryptophan aromatic indole chromophores across adjacent peptides. At low heparin concentrations, no significant change in MAD1's CD signal is observed (FIG. 2a, left), indicating that the glycosaminoglycan is unable to intercalate within tryptophan zippered MAD1 assemblies under these conditions. However, as the weight percent of heparin is increased to be proportional with MAD1 (FIG. 2a, middle), and then in 10-fold excess (FIG. 2a, right), tryptophan-tryptophan interactions are lost and a β-sheet canonical minimum at 212 nm emerges. Parallel studies using thioflavin T (ThT), a dye that increases in fluorescence upon intercalation into β-sheet environments, confirmed the presence of cross β-structures only within HMG assemblies, and not in either mono-formulation (FIG. 2b). Interestingly, in the presence of free heparin, ThT's excitation maximum redshifts to ˜565 nm, suggesting the anionic carbohydrate adsorbs to the cationic dye to generate aggregation-induced ThT excimers.^{[14]} Collectively, our data shows that heparin interleaves between registered tryptophan side chains of MAD1 zippered assembles to yield β-sheet rich hierarchical nano- and micro-structures that ultimately produce HMGs.

Next, we investigated the specificity of heparin-MAD1 binding interactions by performing chromatography-based agglutination assays. Here, MAD1 is incubated with various anionic biomolecules before centrifugal filtration to remove aggregates and quantification of remaining monomer via HPLC analysis. A loss of MAD1 signal indicates avidity for, and agglutination with, the co-incubated biomolecule. Results in FIG. 2c show that the monosaccharides glucose and mannose do not appreciably interact with MAD1, nor does the anionic protein bovine serum albumin (BSA). Together, this suggests that a combination of electrostatics and carbohydrate-π interactions drive MAD1 assembly with heparin, and that neither property alone is sufficient to trigger hierarchical organization. In other words, simply presenting the cationic peptide with an anionic biopolymer, like BSA, will not independently initiate association, and that multivalent carbohydrate-aromatic interactions play an important role in assembly. However, although an anionic polysaccharide is required for assembly, sugar presentation within the carbohydrate appears irrelevant, as MAD1 showed an equivalent assembly propensity with hyaluronic acid (HA) as it does with heparin (see green and orange traces in FIG. 2c).

### Example 2

### Biologic Activity of HMGs

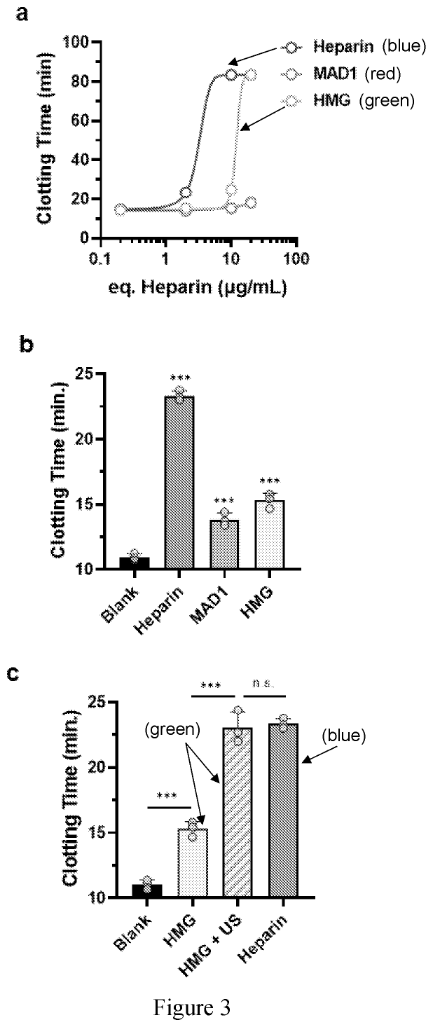

Heparin inhibits coagulation by specifically binding to antithrombin via a sulfated pentasaccharide motif. To evaluate if assembly with MAD1 buries this pentasaccharide sequence, and therefore inhibits heparin's anticoagulant activity, we compared clotting time of blood treated with HMGs, or the individual heparin and MAD1 components as controls (FIG. 3a). Results confirm a marked reduction in heparin's anticoagulant capabilities when complexed with MAD1, requiring nearly an order of magnitude higher equivalent concentration of heparin within HMGs to achieve similar effects to the free agent. Interestingly, we also observed a significant increase in clotting time for blood treated with MAD1 alone (FIG. 3b). This suggests that the peptide itself may have anticoagulant behavior. Platelet binding studies, described later, indicate this may be due to MAD1's propensity to bind to the surface of activated platelets. Nevertheless, despite anticoagulant functions of both components, assembly of heparin with MAD1 to form HMGs appears to significantly reduce its anti-clotting activity.

Yet, this behavior provides an opportunity for HMGs to effectively ‘turn-on’ heparin bioactivity under mechanical stimuli experienced during clot-induced blood flow disruptions. Here, HMGs that diffuse from their subcutaneous depot and into blood circulation will experience high fluid shear forces at sites of thrombosis.^{[15-17]} This may allow for local activation of heparin-MAD1 complexes by decoupling the polysaccharide from the peptide carrier. To test this, we evaluated the anticoagulant activity of HMGs after exposure to ultrasound sonication (40 kHz, 240 W) intended to model mechanical forces experienced during perturbed cardiovascular flow (FIG. 3c). Mechanical decoupling of heparin and MAD1 restored the anticoagulant potency of the polysaccharide.

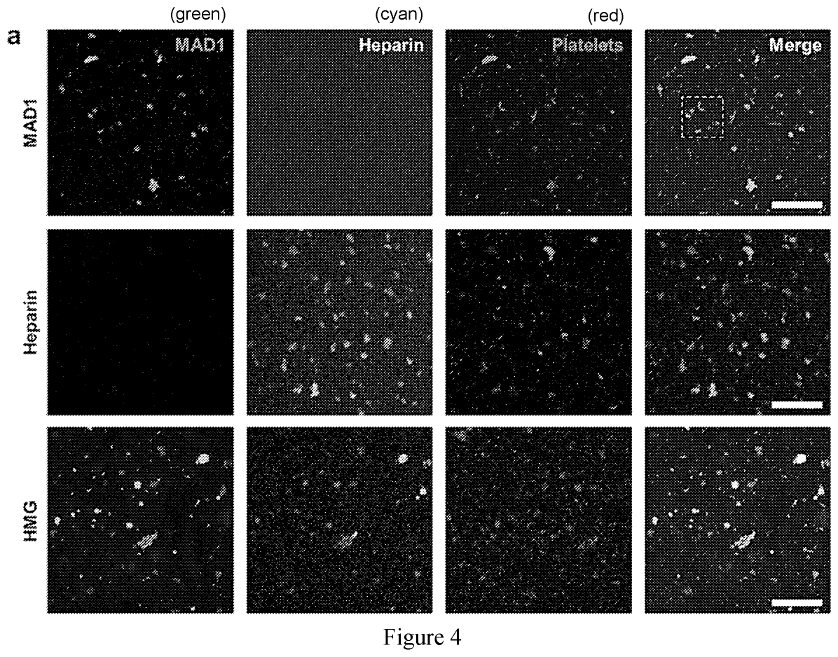

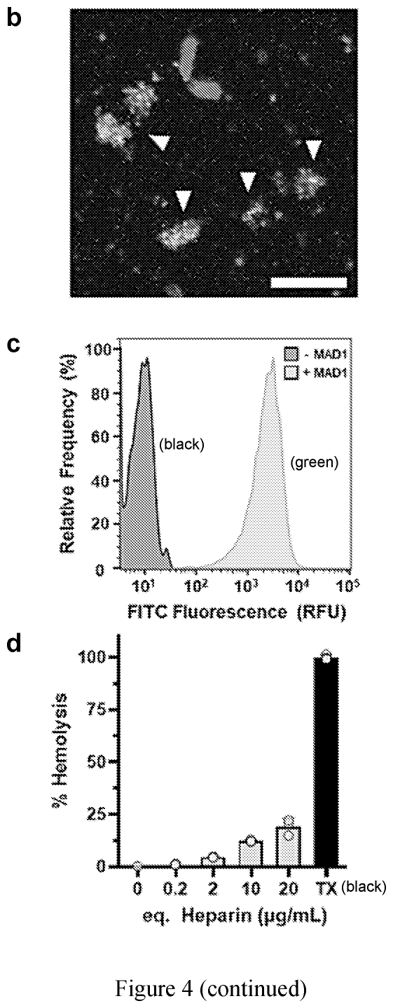

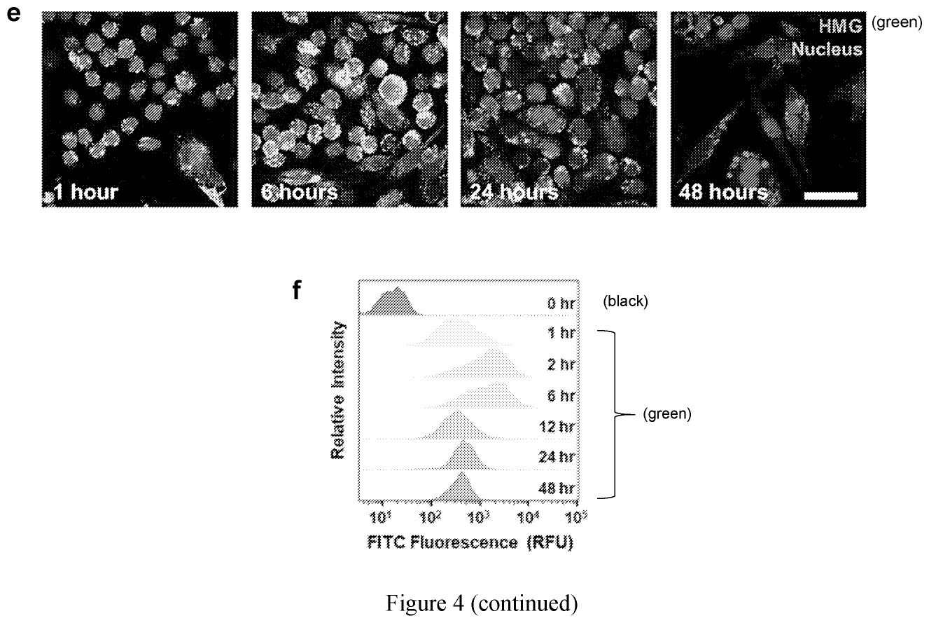

We next employed multicolor confocal microscopy to study the binding of HMGs, as well as the individual heparin and MAD1 components, to activated platelets (FIG. 4a). Confocal micrographs of MAD1-treated samples demonstrate that the peptide rapidly adsorbs to the surface of activated platelets, generating dense cell clusters (FIG. 4b). Parallel flow cytometry assays confirmed the high avidity of MAD1 for activated platelets (FIG. 4c), suggesting that localization of the peptide to the cell surface may trigger peptide fibrillization and long-range platelet accretion. These interactions are likely mediated by changes in the surface charge of platelets upon activation. During clotting, anionic lipids translocate from the inner to outer leaflet of platelets, with additional transbilayer movement generating procoagulant vesicles rich in negatively charged phospholipids.^{[18-20]} Collectively, this leads to a highly anionic surface charge that likely recruits positively charged MAD1 protofibrils. Extrapolating further, this suggests that MAD1 peptides may preferentially target the surfaces of platelets within clots, and thereby direct HMGs to actively growing thrombi. Confocal micrographs shown in FIG. 4a confirm the ability of HMGs to co-localize with activated platelets.

Finally, in preparation for animal studies, we tested the in vitro safety and clearance of HMGs by performing hemolysis and macrophage uptake assays, respectively. Results in FIG. 4d show that the granules are generally well tolerated by erythrocytes, with <15% hemolysis at concentrations≤10 μg/mL of equivalent heparin. To predict the potential mode(s) of in vivo clearance for HMGs, we also evaluated the uptake and degradation of the granules within macrophages. Due to the large size of HMGs (˜200 nm), the particles are expected to show slow renal filtration and urinary clearance, instead being phagocytosed by circulating macrophages and liver Kupffer cells. To evaluate this, we performed time dependent fluorescence microscopy (FIG. 4e) and flow cytometry (FIG. 4f) assays on HMG treated RAW264.7 (−)NO murine macrophages. Our results show that macrophages rapidly phagocytose HMGs, achieving peak uptake around 2 hours of exposure. The granules are subsequently trafficked to the phagolysosome, where they remain up to 48 hours. This long persistence of HMGs within circulating macrophages may help to prolong therapeutic concentrations of heparin in plasma. Additionally, heparin suppresses the expression of proinflammatory cytokines in macrophages and promotes their polarization to anti-inflammatory phenotypes.^{[21, 22]} In fact, flow cytometry results suggest that HMG-treated RAW264.7 (−)NO macrophages display scattering profiles indicative of an anti-inflammatory M2 phenotype.^{[23]} This suggests that, in addition to their ability to sustain anticoagulant responses, HMGs may also serve as long-acting anti-inflammatory agents.

### Example 3

### In Vivo HMG Distribution and Anticoagulant Efficacy

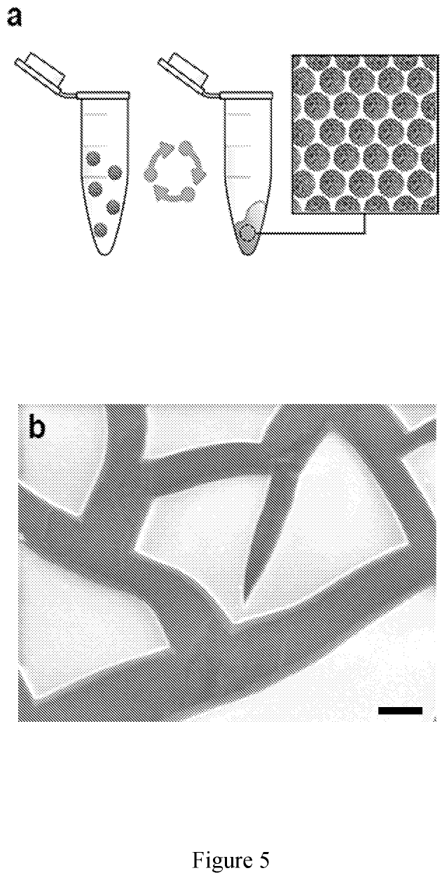

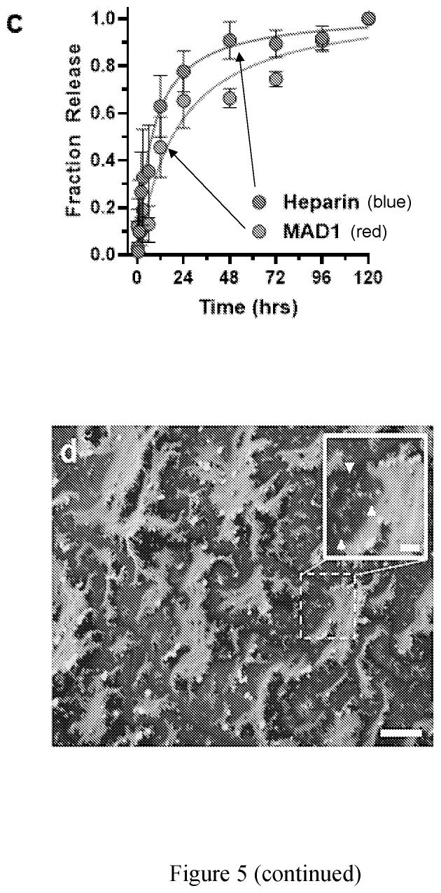

Before initiating in vivo studies, we first optimized the centrifugal compaction of HMGs to form a dense injectable paste (FIG. 5a). Scanning electron microscopy performed on the compacted material shows a densely packed gel is formed after ultracentrifugation (FIG. 5b). Release studies using differentially labeled heparin and MAD1 show first-order release of both HMG constituents, achieving 50% release within 12 hours and 90% release at approximately 84 hours of incubation. Similar release kinetics for heparin and MAD1 suggest that both components are liberated from the eroding paste as HMGs, and not as free agents. Fluorescent microscopy studies of the paste during degradation −+support this assertion and show micron sized granules being released from the eroded material (see white arrows in FIG. 5d, inset).

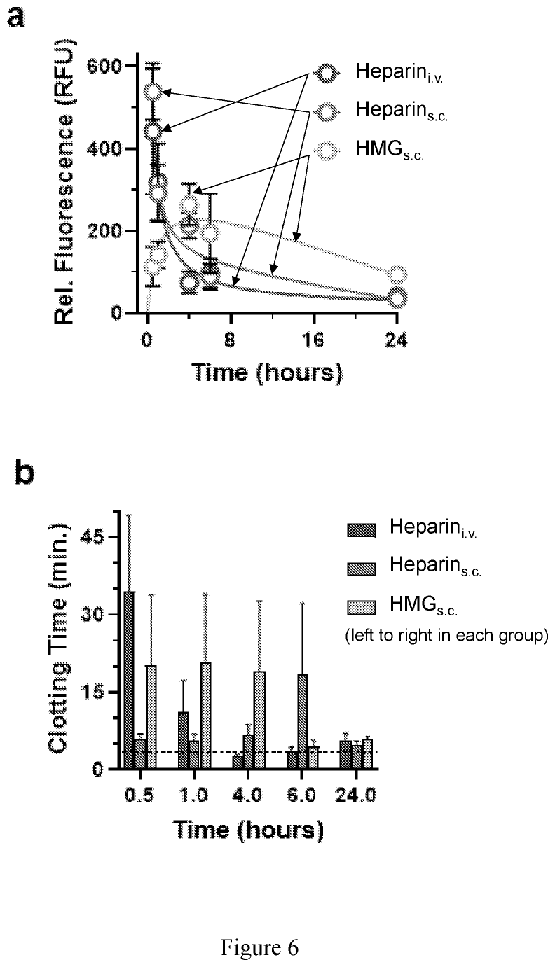

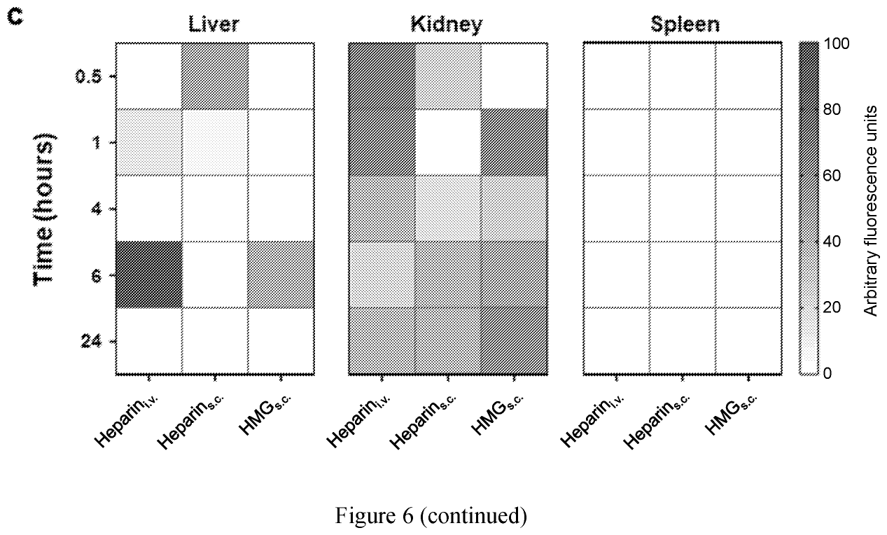

Next, we performed time-dependent in vivo biodistribution and anticoagulation assays in C57BL/6J mice receiving a single subcutaneous dose of HMGs. Two additional groups received an equivalent dose of free heparin given intravenously (i.v.) or subcutaneously (s.c.) as standard clinical care and mode of administration controls, respectively. Results in FIG. 6a show that, for both the heparin_{i.v.} and heparin_{s.c.} groups, heparin concentration in the serum is the highest at the first sampling timepoint (30 minutes) and quickly declines thereafter, reaching complete clearance at ˜6 hours post administration. The HMG_{s.c.} group showed a markedly different pharmacokinetic profile, with plasma concentrations gradually increasing over 0-6 hours, before slowly declining over time. This led to a heparin serum half-life that was an order of magnitude higher for the HMG_{s.c.} group (t_{1/2}=19.2 h) compared to heparin_{i.v.} (t_{1/2}=1.3 h) and heparin_{s.c.} (t_{1/2}=1.4 h) conditions.

Results from parallel blood clotting time assays followed similar kinetic trends (FIG. 6b). For example, clotting time for heparin_{i.v.} treated animals decreased from 35.5 minutes at the 0.5 hour time point, to basal clotting activity (3.5 minutes, dashed line in FIG. 6b) within approximately 2 hours post administration. Heparin_{s.c.}, on the other hand, showed negligible changes in clotting behavior for all time points except 6 hours. Close inspection of the half-life curves in FIG. 6a show that heparin_{s.c.} groups experience a transient spike in heparin serum concentration 4-6 hours after dosing. This suggests that subcutaneously delivered heparin may experience a delayed release phase, where a bolus dose of the anticoagulant reaches circulation following its diffusion through the sub-dermal layer. This rapid and uncontrolled systemic delivery is exemplary of the pharmacokinetic liabilities of free heparin that contribute to adverse bleeding events.^{[24, 25]} In contrast, plasma collected from HMG_{s.c.} animals showed persistent and controlled anticoagulant responses, achieving consistent clotting times of 20 minutes across the 0.5-4 hour sampling time points. Normal clotting behavior was restored at 6 hours post HMG administration.

Finally, we compared tissue biodistribution profiles of heparin in each treated animal group, focusing on the canonical heparin clearance organs: liver, kidneys and spleen.^{[26]} Results in FIG. 6c show that intravenous heparin (heparin_{i.v.}) is initially excreted through the renal system, with additional hepatic clearance observed at ˜6 hours. Heparin_{s.c.} followed an inverse trend, with early liver uptake that later shifted to renal clearance. The HMG_{s.c.} group showed prolonged and sustained heparin kidney clearance, with a similar kinetic profile to serum persistence (FIG. 6a). None of the formulations resulted in significant heparin accumulation within the spleen, despite our in vitro observations of robust HMG macrophage uptake (FIG. 4e,f).

The pharmacologic liabilities of heparin anticoagulants, which include rapid renal clearance, short half-life and adverse effects, as well as clinical barriers that include dosing errors and patient compliance, have made these compounds attractive candidates for biomaterials-enabled delivery strategies. The foregoing Examples demonstrate a robust, and scalable peptide-based heparin delivery technology with the potential to minimize the need for post-administration monitoring, extend therapeutic plasma concentrations of the anticoagulant, and reduce off-target distribution and adverse bleeding events. Parallel strategies exploiting other bio-macromolecular carriers, including proteins^{[27, 28]} and even whole cells,^{[29]} have similarly demonstrated how the intrinsic bioactivity of the vehicle can enhance the pharmacokinetic profile, therapeutic precision and safety of anticoagulant and thrombolytic cargo. However, most of these systems still rely on intravenous administration routes to be operational, thereby increasing the likelihood of dosing errors and limiting their utility to clinical settings. Creation of easy-to-administer subcutaneous heparin formulations, like the HMG materials described here, addresses these pharmacologic hurdles and open new opportunities for effective anticoagulant use in non-clinical settings.

### Example 4

This Example provides a description of materials and methods used in the Examples above.

### Materials

Rink amide ProTide resin and Fmoc-protected amino acids were purchased from CEM (Matthews, NC). Biotech CE dialysis tubing (MWCO 300 kDa), 5/6-carboxyfluorescein succinimidyl ester (NHS-Fluorescein), ethyl ether, acetonitrile, formic acid (LC-MS grade), bovine serum albumin (BSA), calcium chloride (CaCl₂)), 10× phosphate buffer saline (PBS), 1×PBS, sodium chloride (NaCl), and ProLong Diamond Antifade Mountant with DAPI were purchased from ThermoFisher Scientific (Waltham, MA). Trifluoroacetic acid (TFA) was purchased from Chem-Impex Int. (Wood Dale, IL). Thioanisole, heparin sodium salt from porcine intestinal muscoa (IU>=100/mg), and DiI were purchased from Alfa Aesar (Haverhill, MA). 1,2-Ethanedithiol and mannose were purchased from Acros Organics (Waltham, MA). Anisole was purchased from TCI chemicals (Tokyo, Japan). 5% mol, substituted Rhodamine-tagged heparin (rhodamine-Hep) and 1% mol, substituted aminomethyl coumarin-tagged heparin (AMCA-Hep) were purchased from HAWorks (Bedminster, NJ). Glucose and Triton-X100 were purchased from Sigma-Aldrich (St. Louise, MO). Sodium hyaluronate 100K was purchased from Lifecore Biomedical (Chaska, MN). Sodium phosphate dibasic, sodium phosphate monobasic, Tris, and L-glutamine were purchased from VWR (Radnor, PA). Thioflavin T (ThT) was purchased from Oakwood Chemicals (Estill, SC). Citrated adult bovine whole blood was purchased from Lampire Biological Laboratory (Pipersville, PA). RPMI-1640 medium was purchased from Lonza (Basel, Switzerland). Fetal bovine serum (FBS) was purchased from Hyclone Laboratories Inc (Logan, UT). 4% Paraformaldehyde (PFA) was purchased from ChemCruz (Dallas, TX). RAW 264.7 (−)NO murine macrophage cell was a gift from Dr. Yong Wang's laboratory at the Pennsylvania State University Department of Biomedical Engineering.

### Peptide Synthesis

MAD1 peptide was synthesized as described previously.^{[9]} In brief, Fmoc-based solid-phase peptide synthesis was performed on Rink Amide ProTide Resin using CEM Liberty Blue Automated Microwave Peptide Synthesizer. Fluorescent labeling of MAD1 using NHS-Fluorescein (FITC-MAD1) was performed as previously described. Next, peptide cleavage and deprotection was performed by stirring in TFA:thioanisole:1,2-ethanedithiol:anisole (90:5:3:2 ratio) solution under argon for 3 hours at 40° C. Peptides were purified by reverse-phase HPLC (Shimadzu; Columbia, MD) using a Phenomenex Luna Omega PS C18 column (Torrance, CA) with a linear 1%/min. solvent gradient of solvent A (0.1% TFA) and solvent B (0.1% TFA in 90% acetonitrile). Purity of MAD1 peptide was confirmed by reverse-phase HPLC-MS. Purified MAD1 was lyophilized and stored at −20° C. before use.

### HMG Synthesis

HMG was synthesized by an electrospray method previously developed in our lab.^{[11, 31]} Briefly, heparin and MAD1 peptide were dissolved in de-ionized (DI) water at 1 mg/mL to create the spray and bath solutions, respectively. Before electrospray, 1 mL of MAD1 bath solution was further diluted in 8 mL DI water. The heparin spray solution was loaded into a 5 mL syringe coupled with a 0.5-inch 28G stainless steel needle (Hamilton; Reno, NV) charged to 24 kV using a high voltage power supply (Spellman; Hauppauge, NY). 1 mL (at 0.1 mL/min) of the heparin solution was then spraying into the MAD1 bath. The final product consisted of 1 mg Heparin and 1 mg MAD1 with a total volume of 10 mL. Fluorescently labelled HMGs were prepared using AMCA or rhodamine tagged heparin as the electrospray solution and MAD1 bath containing 10 vol % of FITC-MAD1. After synthesis, HMG solutions were incubated on a shaker for 1 hour at 37° C. to allow for complete assembly. The unreacted components were removed via dialysis (300 kDa MWCO membrane) overnight. HMG particles were stored at 37° C. until use.

### HMG Characterization

HMG particle size and surface charge were determined by dynamic light scattering and zeta potential measurements, respectively, using a Malvern Zetasizer Nano ZS (Malvern, PA). HMG samples were diluted 10 vol % in DI water and loaded into disposable cuvette before taking three independent measurements (25° C., 175° scattering angle). Scanning electron microscopy (SEM) of HMG was performed using a Zeiss SIGMA VP-FESEM (Oberkochen, Germany). Samples were prepared by adding a 10 μL of the particle solution onto stubs and dried at 37° C. SEM imaging was performed at 10 kV landing energy in variable pressure (VP) mode.

Circular dichroism (CD) assays were performed by preparing 0.2 mg/mL MAD1 in PBS, and diluting with an equal volume of 0.02-2 mg/mL heparin solution in PBS to a total sample volume 200 μL. After incubation for 1 hour to allow assembly, the mixture was transferred to a 10 mm path length quartz CD cuvette cell and ellipticity measured at 25° C. over 185-260 nm wavelengths using a Jasco J-1500 Circular Dichroism Spectrometer (Jasco; Oklahoma City, OK). Three independent measurements were taken, and CD spectra reported as average ellipticity (mdeg).

Thioflavin T (ThT) fluorescent assays were performed by preparing 0.2 mg/mL heparin in DI water and diluted with an equal volume of MAD1 at different concentrations, or DI water as a negative control. 100 μL of the solution was transferred to a 96-well black plate and concentrated ThT solution added to each well to achieve 50 μM final ThT concentration. Fluorescence spectra over 460-700 nm was collected at an excitation of 430 nm using BioTek Cytation 3 Microplate Reader (Winooski, VT).

Reverse phase LC-MS binding assays were performed by preparing solutions of glucose, mannose, BSA, hyaluronic acid, or heparin in LC-MS grade water at 2 mg/mL. An equal volume of a 2 mg/mL MAD1 solution was mixed in a microtube, incubated at 37° C. for 30 minutes and centrifuge filtered (0.2 μm) to remove precipitates. Analytical reverse phase LC-MS was performed using a gradient of 0-100% solvent B over 50 minutes (40° C.) through a Phenomenex Luna C18 column. Data is presented by plotting ESI+ (505 m/z) mass spectrometry TIC.

### Clotting Time Assay

Hemostatic activity of HMG was evaluated by clotting time assays using bovine platelet rich plasma (PRP). Here, bovine PRP was acquired from the top clear layer after centrifuging citrated bovine whole blood at 600 rcf for 30 minutes (4° C.) with minimum acceleration/break ramps. Next, HMG treatment solutions (10×) were prepared by serially diluted freshly prepared HMG in DI water to a final concentration of 0.2-20 μg/mL equivalent heparin. For mechanical-associated decoupling of HMG, the particle solution (10 μg/mL equivalent heparin) was sonicated (40 kHz, 240 W) for 5 minutes prior to the clotting assay. Clot formation was initiated by combining 450 μL bovine PRP, 50 μL HMG treatment solution, and 50 μL 0.1M CaCl₂) in a 24-well plate. DI water was included as a negative control. Samples were mixed on an oscillating rocker plate and clotting time determined as the time required for observable aggregate formation upon recalcification.

### HMG Platelet Localization

Bovine platelets were acquired from bovine PRP by centrifugation (800 rcf, 15 minutes), labelled with DiI (5 μg/mL), washed, and resuspended in PBS. At the time of treatment, platelets were further centrifuged (800 rcf, 15 minutes) to replace PBS supernatant with treatment solutions. Treatments of AMCA/FITC HMG, FITC-MAD1, or AMCA-heparin were prepared in PBS at equivalent concentrations, incubated with platelet samples for 15 minutes and then transferred to 4-well chambered slide (500 μL) before imaging using an Olympus FluoView 1000 confocal microscope (Tokyo, Japan).

MAD1-platelet binding was analyzed via flow cytometry by resuspending platelets in PBS containing 1 mg/mL FITC-MAD1. Blank PBS was used as an untreated control. After a 15-minute incubation, treated platelets were centrifuged (800 rcf, 15 minutes) and resuspended in PBS before analysis using a Guava Flow Cytometer (Millipore Sigma; Burlington, MA). Data was analyzed by FlowJo software.

### HMG Hemolysis

Human RBCs were acquired via whole blood centrifugation (600 rcf, 30 minutes), washed in PBS and resuspended (0.25 vol %) in hemolysis buffer (10 mM Tris, 150 mM NaCl, pH 7.4). HMG sample solutions were serially diluted in hemolysis buffer to 0.2-20 μg/mL equivalent heparin. Blank hemolysis buffer or 1% Triton-X100 were included as negative and positive controls, respectively. To start the hemolysis assay, 75 μL of the RBC solution was mixed with an equivalent volume of the HMG solution in a 96-well plate, incubated overnight and then centrifuged (4000 rpm, 10 minutes, 4° C.) to remove intact RBCs. 100 μL of supernatant was transferred to a clean 96-well plate and absorbance read at 415 nm using a microplate reader. % hemolysis was calculated as followed:

$$\% hemolysis = \frac{{absorbance}_{treatment} - {absorbance}_{negative}}{{absorbance}_{positive} - {absorbance}_{negative}}$$

### Macrophage Uptake

RAW 264,7 (−)NO murine macrophages were cultured in RPMI-1640 supplemented with 10 vol % FBS and 2 mM L-glutamine. Cell were incubated at 37° C. and 5% CO₂ before use. RAW 264.7 (−)NO cells were seeded on 4-well chambered slides (20,000 cells/well) and incubated overnight to adhere. HMGs prepared using rhodamine-labeled heparin and FITC-labeled MAD1 were added to the culture media to a final concentration of 10 μg/mL equivalent heparin. After incubation, treated cells were washed with PBS, fixed with 4% PFA (15 minutes), and mounted using Antifade ProLong Diamond Mountant with DAPI before imaging using an Olympus FluoView 1000 confocal microscope. Cell fluorescence was analyzed by ImageJ software. Similar experimental conditions were used for flow cytometry, with RAW 264.7 (−)NO cells being seeding onto 12-well plates at 100,000 cells/well before treatment with rhodamine/FITC HMGs. At various time points cells were removed and flow cytometry performed using a Guava Flow Cytometer. Data was analyzed by FlowJo software.

### HMG Paste Preparation

Particles were prepared using rhodamine- and FITC-labeled heparin and MAD1, respectively. To prepare the HMG paste for injection into animal models, particle solutions were centrifuged at 20,000 rcf for 5 seconds. Characterization of the HMG pellet was performed by transferring ˜10 μL aliquot of the samples to a stub or glass slide for SEM or fluorescent microscopy analysis, respectively. For SEM, samples were dried in a 37° C. oven and imaged using Zeiss SIGMA VP-FESEM variable pressure mode at 10 kV landing energy. Epifluorescent microscopy was performed on hydrated samples using a BioTek Cytation 3 Plate Imager. Disassociation and release of HMG particles from the paste incubated in PBS buffer was quantified via fluorescence measurements. To initiate release, compacted HMG was acclimated in PBS buffer. At each timepoint, 100 μL of supernatant was transferred to a 96-well plate and measured at λₑₓ=540 nm, λₑₘ=570 nm (rhodamine) and λₑₓ=490 nm, λₑₘ=525 nm (FITC) using a BioTek Cytation 3 Plate Reader. Fraction release was calculated by normalizing to the equilibrium value after 120 hours of the experiment, with the release profile adjusted for mass loss during sample collection.

### Animal Studies

Murine studies were performed under IACUC protocol 202102036. HMG particles were prepared using rhodamine-heparin doped 20 vol % into unlabeled heparin before electrospray synthesis. Dialyzed HMG particles were concentrated by Centri Vap DNA Concentrator to achieve 0.5 mg/mL equivalent heparin. Sterile 10×PBS was diluted ten-fold into samples before injection of a 200 μL volume into C57BL/6J mice. Two groups (n=4) received free heparin given via tail vein injection or injected subcutaneously in the neck scruff. A third group (n=4) received HMG sample as a subcutaneous depot in the neck scruff. All mice received a final heparin dose of 10 IU. At each time point, mice were sacrificed, and blood samples collected into citrate tubes via cardiac puncture. Coagulation time was determined on whole blood samples via INTEM analysis on a ROTEM delta coagulation analyzer (Werfen; Bedford, MA). Clotting times were chosen to evaluate the efficacy of each treatment's ability to inhibit thrombin-linked platelet activation and initial fibrin polymerization/recruitment. INTEM coagulation analysis was selected based on its established sensitivity for the presence of heparin. For a typical experiment, serum is isolated via centrifugation (1000 rcf, 10 minutes) and fluorescence measurements taken (λₑₓ=560 nm, λₑₘ=590 nm). Kidney, liver, and spleen tissues were also collected and homogenized before centrifugation at 10,000 rcf for 20 minutes to remove bulk fibrous structures. 100 μL of the remaining supernatant was evaluated for heparin presence via fluorescence measurements (λₑₓ=560 nm, λₑₘ=590 nm) using a BioTek Cytation 3 Plate Reader.

References—this reference listing is not an indication that any reference is material to patentability
1. Harenberg, J.; Huhle, G.; Piazolo, L.; Giese, C.; Heene, D. L. In Long-term anticoagulation of outpatients with adverse events to oral anticoagulants using low-molecular-weight heparin, Seminars in Thrombosis and Hemostasis, 1997; pp 167-172.
2. Magnani, H. N.; Gallus, A., Thromb. Haemostasis 2006, 95 (06), 967-981.
3. Newall, F.; Johnston, L.; Ignjatovic, V.; Monagle, P., Pediatrics 2009, 123 (3), e510-e518.
4. Sanson, B.-J.; Lensing, A. W.; Prins, M. H.; Ginsberg, J. S.; Barkagan, Z. S.; Lavenne-Pardonge, E.; Brenner, B.; Dulitzky, M.; Nielsen, J. D.; Boda, Z., Thromb. Haemostasis 1999, 81 (05), 668-672.
5. Liang, Y.; Kiick, K. L., Acta Biomater. 2014, 10 (4), 1588-1600.
6. Paliwal, R.; Paliwal, S. R.; Agrawal, G. P.; Vyas, S. P., Med. Res. Rev. 2012, 32 (2), 388-409.
7. Das Kurmi, B.; Tekchandani, P.; Paliwal, R.; Rai Paliwal, S., Curr. Pharm. Des. 2015, 21 (30), 4509-4518.
8. Qi, Y.; Zhao, G.; Liu, D.; Shriver, Z.; Sundaram, M.; Sengupta, S.; Venkataraman, G.; Langer, R.; Sasisekharan, R., Proc. Natl. Acad. Sci. 2004, 101 (26), 9867-9872.
9. Simonson, A. W.; Mongia, A. S.; Aronson, M. R.; Alumasa, J. N.; Chan, D. C.; Lawanprasert, A.; Howe, M. D.; Bolotsky, A.; Mal, T. K.; George, C., Nat. Biomed. Eng. 2021, 5 (5), 467-480.
10. Abreu, R.; Essler, L.; Loy, A.; Quinn, F.; Giri, P., Sci. Rep. 2018, 8 (1), 1-12.
11. Simonson, A. W.; Umstead, T. M.; Lawanprasert, A.; Klein, B.; Almarzooqi, S.; Halstead, E. S.; Medina, S. H., Biomaterials 2021, 273, 120848.
12. Cochran, A. G.; Skelton, N. J.; Starovasnik, M. A., Proc. Natl. Acad. Sci. 2001, 98 (10), 5578-5583.
13. Liu, J.; Yong, W.; Deng, Y.; Kallenbach, N. R.; Lu, M., Proc. Natl. Acad. Sci. 2004, 101 (46), 16156-16161.
14. Sulatskaya, A. I.; Lavysh, A. V.; Maskevich, A. A.; Kuznetsova, I. M.; Turoverov, K. K., Sci. Rep. 2017, 7 (1), 2146-2146.
15. Fogelson, A. L.; Neeves, K. B., Annu. Rev. Fluid Mech. 2015, 47, 377-403.
16. Zakaria, M. S.; Ismail, F.; Tamagawa, M.; Azi, A. F. A.; Wiriadidjaya, S.; Basri, A. A.; Ahmad, K. A., J Adv. Res. Fluid Mech. Therm. Sci. 2018, 43 (1), 81-89.
17. Shadden, S. C.; Hendabadi, S., Biomech. Model. Mechanobiol. 2013, 12 (3), 467-474.
18. Rumbaut, R. E.; Thiagarajan, P., Synthesis Lectures on Integrated Systems Physiology: From Molecule to Function 2010, 2 (1), 1-75.
19. Peng, B.; Geue, S.; Coman, C.; Münzer, P.; Kopczynski, D.; Has, C.; Hoffmann, N.; Manke, M.-C.; Lang, F.; Sickmann, A., Blood 2018, 132 (5), e1-e12.
20. Prescott, S. M.; Zimmerman, G. A.; Stafforini, D. M.; McIntyre, T. M., Annu. Rev. Biochem. 2000, 69 (1), 419-445.
21. Bruno, V.; Svensson-Arvelund, J.; Ruber, M.; Berg, G.; Piccione, E.; Jenmalm, M. C.; Ernerudh, J., Sci. Rep. 2018, 8 (1), 1-9.
22. Abbadi, A.; Loftis, J.; Wang, A.; Yu, M.; Wang, Y.; Shakya, S.; Li, X.; Maytin, E.; Hascall, V., J. Biol. Chem. 2020, 295 (15), 4849-4857.
23. Kaczmarek, M.; Lagiedo, M.; Masztalerz, A.; Kozlowska, M.; Nowicka, A.; Brajer, B.; Batura-Gabryel, H.; Sikora, J., Immunobiology 2018, 223 (2), 200-209.
24. Hirsh, J.; Warkentin, T. E.; Shaughnessy, S. G.; Anand, S. S.; Halperin, J. L.; Raschke, R.; Granger, C.; Ohman, E. M.; Dalen, J. E., Chest 2001, 119 (1), 64S-94S.
25. Kandrotas, R. J., Clin. Pharmacokinet. 1992, 22 (5), 359-374.
26. Boneu, B.; Caranobe, C.; Sie, P., Bailliere's Clin. Haematol. 1990, 3 (3), 531-44.
27. Wen, A. M.; Wang, Y.; Jiang, K.; Hsu, G. C.; Gao, H.; Lee, K. L.; Yang, A. C.; Yu, X.; Simon, D. I.; Steinmetz, N. F., J. Mater. Chem. B 2015, 3 (29), 6037-6045.
28. Pitek, A. S.; Park, J.; Wang, Y.; Gao, H.; Hu, H.; Simon, D. I.; Steinmetz, N. F., Nanoscale 2018, 10 (35), 16547-16555.
29. Chen, C.; Li, S.; Liu, K.; Ma, G.; Yan, X., Small 2016, 12 (34), 4719-4725.
30. Aronson, M. R.; Dahl, E. S.; Halle, J. A.; Simonson, A. W.; Gogal, R. A.; Glick, A. B.; Aird, K. M.; Medina, S. H., Cell. Mol. Bioeng. 2020, 13 (5), 447-461.
31. Simonson, A. W.; Lawanprasert, A.; Goralski, T. D. P.; Keiler, K. C.; Medina, S. H., Nanomedicine 2019, 17, 391-400.
32. Ofosu, F.; Blajchman, M. A.; Hirsh, J., Thromb. Res. 1980, 20 (4), 391-403.

1. A composition for use in inhibiting blood clotting, the composition comprising a peptide comprising the amino acid sequence KRWHWWRRHWVVW (SEQ ID NO:1), and wherein the peptide optionally comprises an amido group at the C-terminus of the peptide, and a heparin.

2. The composition of claim 1, wherein the composition exhibits a negative zeta potential.

3. The composition of claim 2, wherein the heparin and the peptide are associated with one another by an electrostatic force and carbohydrate-π interactions.

4. The composition of claim 1, wherein the composition comprises granules.

5. The composition of claim 4, wherein the composition is configured for use as a subcutaneously administered formulation.

6. The composition of claim 5, wherein the composition is configured to diffuse from a subcutaneous location into blood of an individual.

7. The composition of claim 6, wherein once diffused into the blood of the individual, the anticoagulant component dissociates from the peptide due to a high fluid shear force in blood of the individual.

8. A method comprising introducing into an individual in need thereof a composition of claim 4.

9. The method of claim 8, wherein the composition is introduced subcutaneously.

10. The method of claim 9, wherein the composition diffuses into the blood stream of the individual.

11. The method of claim 10, wherein the anticoagulant agent dissociates from the peptide due to a high shear force in blood of the individual.

12. The method of claim 11, wherein the high shear force is caused by a clot-induced blood flow disruption, and wherein the anticoagulant agent reduces the clot and/or inhibits further clot formation.

13. The method of claim 11, wherein the subcutaneous introduction extends a therapeutic plasma concentration of the anticoagulant component.

14. The method of claim 13, wherein the individual is in need of the composition due to a surgical procedure, a blood or organ condition that includes a risk of deleterious blood clotting, or the individual is at risk for or has disseminated intravascular coagulation, thrombosis including but not limited to venous thrombosis and deep-vein thrombosis, a pulmonary embolism, peripheral arterial embolism, a stroke, acute coronary syndrome, atrial fibrillation, a heart attack, unstable angina, or wherein the individual is undergoing prolonged bed rest or hemofiltration, or has an indwelling catheter, or any combination of the foregoing.
